(19) United States
(12) Patent Application Publication
(10) Pub. No.: US 2025/0352289 A1
(43) Pub. Date: Nov. 20, 2025

(54) INTEGRATED MULTI-ARM MOBILE MODULAR SURGICAL ROBOTIC SYSTEM

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 19/288,723

(22) Filed: Aug. 1, 2025

Related U.S. Application Data
(63) Continuation of application No. PCT/EP2024/052353, filed on Jan. 31, 2024.
(60) Provisional application No. 63/444,988, filed on Feb. 12, 2023.

(51) Int. Cl. A61B 34/35; A61B 34/30; A61B 50/13; B25J 13/06

(52) U.S. Cl. CPC A61B 34/35; A61B 50/13; B25J 13/06; A61B 2034/301; A61B 2034/302

(57) ABSTRACT

A surgical robotic system includes two or more mobile robotic carts that can be separably joined together in a selected, fixed pattern and deployed at least partially beneath a surgical table. The mobile carts carry robotic arms that are controlled by a common controller in a common surgical coordinate system. By selecting separation distances between individual robotic carts, the robotic system can be arranged to perform different surgical procedures.

### CROSS-REFERENCE

This application is a continuation of PCT Application No. PCT/EP2024/052353, filed Jan. 31, 2024, which claims the benefit of U.S. Provisional Application No. 63/444,988, filed Feb. 12, 2023, each of which are incorporated herein by reference.

### BACKGROUND

### Field

The disclosed technology relates to robotic surgical apparatus, systems, and methods. More particularly, the disclosed technology relates to mobile, bilateral surgical robotic systems comprising multiple mobile carts that can be interconnected to form an integrated surgical platform having a shared robotic coordinate system.

Robotic surgery has been adapted in a variety of surgical procedures, including general surgery and spinal surgery. Many robotic surgery systems, such as the da Vinci robotic surgery system from Intuitive Surgical®, are “teleoperated” from a remote station. Multi-arm robotic surgical systems are available from some vendors but are often also teleoperated and limited to a single arm deployed separately on a separate cart with a remotely positioned control unit. The single arms are usually configured to operate in separate robotic coordinate spaces.

Systems comprising multiple arms on multiple carts have significant drawbacks regarding integration into surgical workflow, along with an undesirably large footprint in the operating room. Also, in cases where these multi-arm systems do utilize a single cart, control of the individual arms is coordinated by a physician who “closes the loop” with his eyes and hands while sitting at a remotely located control unit. Such remote control does not provide the level of control required for many surgical procedures. Accuracy will inevitably be inferior to a system where all robotic arms are fixed to, and coordinated by, one or more single rigid chassis mechanically and accurately joined to each other and comprising a central control unit.

### SUMMARY

In a first aspect, the disclosed technology provides a modular surgical robotic system for performing robotic surgery on a patient lying on a surgical. The system comprises a first robotic chassis and a second robotic chassis, wherein said first and second robotic chassis are configured to be separably joined to form an integrated robotic platform having a common robotic coordinate system relative to the surgical bed. One or more robotic arms are disposed on each of the first rigid and second robotic chassis, and a controller is configured to robotically coordinate the movement of each the robotic arms in the common coordinate system.

In some embodiments, the first and second robotic chassis are configured to be positioned on opposite sides of the surgical bed and to be separably joined beneath and across a width of the surgical bed. to form the integrated robotic platform.

In some embodiments, the modular surgical robotic systems of the disclosed technology further comprise a third robotic chassis and a fourth robotic chassis configured to be positioned on opposite sides of the surgical bed and to be separably joined beneath and across a width of the surgical bed, said third and/or fourth robotic chassis being further configured to be separably joined to the first and/or the second robotic chassis to form an integrated platform having a common robotic coordinate system for all four robotic chassis.

In some embodiments, the first and second robotic chassis are each configured to be positioned beneath and across a width of the surgical bed and to be separably joined in a longitudinal direction.

In some embodiments, each of the robotic chassis is configured to be moved and repositioned relative to the surgical bed.

In some embodiments, the robotic chassis each comprise rollers that allow the chassis to be moved over a floor.

In some embodiments, the modular surgical robotic system of the disclosed technology further comprise connectors configured to separably join adjacent pairs of robotic chassis in a fixed relationship.

In some embodiments, the connectors comprise electrical conductors for power and/or data transmission between said adjacent pairs of robotic chassis.

In some embodiments, the robotic arms each have a point of origin and wherein the points of origin of at least two of the robotic arms are located at least 80 cm apart from each other, usually at least one meter apart from each other.

In some embodiments, the robotic chassis are configured to be separably joined before deployment under a surgical table.

In some embodiments, the robotic chassis are configured to be separably joined after deployment under a surgical table.

In some embodiments, the robotic arms comprise both surgical arms configured to hold surgical tools and surveillance and/or navigation arms configured to hold sensors.

In a second aspect, the disclosed technology provides a method for arranging and controlling a surgical robotic system to perform robotic surgery on a patient lying on a surgical bed having two sides in a surgery room. The method comprises separably joining a first robotic chassis and a second robotic chassis to form an integrated robotic platform having a common robotic coordinate system where each of the robotic chassis carries robotic arms.

The integrated surgical platform is positioned beneath and across a width of the surgical bed to locate robotic arms on both side of the surgical bed, and movement of the robotic arms in the common robotic coordinate system is controlled using a common controller.

In some embodiments, the first robotic chassis and the second robotic chassis are separably joined prior to positioning the integrated surgical platform beneath and across a width of the surgical bed.

In some embodiments, the methods of the disclosed technology further comprise separably joining and positioning third and fourth robotic chassis to the first and/or the second robotic chassis beneath and on opposite sides of the surgical bed to be part of the integrated platform having a common robotic coordinate system.

In some embodiments, the first and second robotic chassis are each positioned beneath and across a width of the surgical bed and separably joined in a longitudinal direction

In some embodiments, the first robotic and second robotic chassis are separably joined after positioning the integrated surgical platform beneath and across a width of the surgical bed.

In some embodiments, the first robotic and second robotic chassis are separably joined before positioning the integrated surgical platform beneath and across a width of the surgical bed.

In some embodiments, positioning comprises moving the robotic chassis over a floor surface adjacent to the surgical bed.

In some embodiments, wherein the robotic chassis comprise rollers and moving comprises manually pushing the chassis over the floor.

In some embodiments, separably joining the robotic chassis comprises attaching a connector between an adjacent pair of robotic chassis to establish a fixed positional relationship.

In some embodiments, attaching the connectors further establishes power and/or data transmission between adjacent pairs of robotic chassis.

In some embodiments, the methods of the disclosed technology further comprise detaching the connector to allow the robotic chassis to be moved separately over the floor.

In some embodiments, the robotic arms each have a point of origin and the first second robotic chassis are separably joining so that the points of origin of at least two of the robotic arms are located at least 80 cm apart from each other, usually at least one meter apart from each other.

In some embodiments, the robotic arms comprise both surgical arms configured to hold surgical tools and surveillance and/or navigation arms configured to hold sensors.

In some embodiments, the methods of the disclosed technology further comprise selecting at least one of an axial separation and a lateral separation between the first robotic chassis and the second robotic chassis, where the first robotic chassis and the second robotic chassis are separably joined at said selected axial separation and/or lateral separation.

In some embodiments, selecting the at least one of an axial separation and a lateral separation between the first robotic chassis and the second robotic chassis comprises determining optimum axial and/or lateral separations for a particular procedure.

The disclosed technology thus can provide an integrated surgical robotic system comprising a plurality of mobile chassis or carts, with each chassis or cart incorporating at least two surgical robotic arms configured to be separably joined and arranged on opposite sides of a surgical table where each of the mobile carts may be partially of fully deployed under the surgical table, either before or after joining. For example, two or more mobile carts may be separably joined to each other, with a controller located on or in one of the carts. The controller may optionally include a display and/or interface to allow interaction while the surgeon is bedside. The controller controls the operation of all of the robotic arms within a common robotic surgical coordinate system which is defined with reference to the common platform of rigidly affixed carts or chassis. Such a common robotic surgical coordinate system allows the controller to perform highly accurate kinematic control of the robot arms without the need to rely on optical (camera-based) real time tracking, although such optical tracking may be performed in addition to kinematic tracking and/or may be used to initially register the patient within the common robotic surgical coordinate system.

The integrated surgical robotic system can support multiple robotic elements, such as robotic arms, end effectors, cameras, imaging devices, tracking devices, and/or other devices useful for robotic surgery. In some embodiments, placement and movement of the robotic elements are kinematically controlled and coordinated by the single, common controller. When each of individual chassis or carts are interconnected, the controller can kinematically control all robotic elements in the single robotic coordinate system which is formed.

Optionally, such kinematic control can be augmented with optical- and sensor-based robotic navigation technology, including external cameras, endoscopic cameras, and the like. For example, any one or more of the chassis that are joined to each other may have two surgical robotic arms and a further robotic navigation or surveillance arm to provide navigation or imaging elements in the surgical field. As noted previously, by joining multiple chassis in a fixed relationship, the robotic arms are located in a single coordinate system and the controller can provide robotic coordination of the surgical arms and the navigation capabilities.

In some embodiments, the disclosed technology provides surgical robotic systems suitable for surgical applications requiring the operation of multiple robotic arms, where there may be two or more surgical arms located “bilaterally,” e.g., on opposite side of a surgical table with two or more navigation or other cameras deployed on additional robotic arms. The robotic arms located on two or more mobile carts are maintained in a fixed relationship to each other with each arm having a point of origin spaced from the point of origin of each of the other arms. By spacing the apart, surgeon access, including visibility and reachability, can be improved. Alternatively, in some embodiments, it may be desirable to keep the robot arms in a tightly packed configuration. The disclosed technology accommodates both situations.

In some embodiments, the disclosed systems are “bilateral,” meaning that one or more robotic arms can extend from a cart or chassis located on each side of a surgical table. The bilateral robotic surgical system comprises at least two chassis or carts, e.g., mobile carts, that are configured to be selectively placed and/or separably joined under the surgical table, where the carts can be joined together before or after placement under the surgical table. In some embodiments, each mobile cart is configured to have ends positioned on opposite sides of the surgical table when the cart extends beneath and across a width of the table. In such instances, the at least two carts will be separably joined in a longitudinal direction with respect to the surgical table. In some embodiments, each mobile cart will be configured to be positions on one side of the table and the two carts separably joined by a connector located beneath and extending across a width of the table.

In some embodiments, the mobile carts will have a lower profile than conventional surgical robots since robotic arms will be configured to be folded down to allow advancement of the cart beneath the table, sometimes being foldable inside the cart. The low profile of the mobile carts and the ability to fold the arms inside the carts or to its side provides for the optional deployment of the mobile carts under the surgical table. This is a critical capability when it comes to saving space in the operating room and to not having the system, its cables and its arms interfere with surgeon workflow.

The mobile carts are designed for use with short, light robotic arms. As there are multiple arms on each side of the patient, all patient anatomy can be reached with shorter arms than a typical one arm or unilateral multiple arm system would require. Short, light robotic arms are usually more accurate and easily maneuverable than larger robotic arms found on many conventional surgical robotic systems. Systems having only a single working robotic arm need to cover a much larger surgical field than the smaller arms of the present system, wherein each arm need access only a portion of the whole surgical filed. Moreover, it is known in the art that a single robotic arm stretched to the farthest extent of its reach is less accurate in carrying out tasks than the same robotic arm used in a more folded deployment. Tin addition to enhanced accuracy, the disclosed technology enables a wide variety of bilateral robotic procedures which would be difficult or impossible to perform with single-arm or unilateral arm surgical robotic systems.

In some embodiments, the robotic arms originate from an integrated surgical robotic platform sharing a common robotic surgical coordinate system. While that could be said of known multi-arm surgical robots located on a single cart or chassis, separable joining of two or more mobile carts allows the bases (points of origin) of at least some of the individual surgical arms to be more widely spaced-apart. The spacing of the points of origin of two or more of the robotic surgical arms may be at least 80 cm, often being one meter, or longer, providing enhanced kinematic flexibility.

In some embodiments, the robotic surgical systems of the disclosed technology may comprise two separate mobile carts configured to be located on opposite sides of the surgical table and to be separably joined across and beneath the surgical table. In some embodiments, appropriate mechanical and electrical connections allow the two constituent mobile units to form integrated robotic platform. Each individual mobile unit carries one or more robotic arms deployable from a side and/or top of the unit. Typically, at least one of one of the mobile units carries the controller which controls the deployment and movement of all the robotic arms by way of the mechanical and electrical connections between the constituent mobile units, e.g., in a primary-secondary control arrangement. In this way, a single chassis mobile robotic surgical system is created from two individual carts and may be “centrally” controlled by a single controller. Of course, for purposes of inventory and uniformity, it may be desirable to construct all the mobile carts to include a controller, user display, user interface, and the like, where only one of the controllers is active while the other(s) is/are inactivated when multiple carts are joined.

The robotic systems of the disclosed technology may optionally further comprise a robotically controlled surgical navigation and/or imaging capability to augment the integrated multi-arm mobile bilateral robotic surgical system. Specifically provided herein, in some embodiments, is a further robotic arm on each of the constituent mobile carts of the integrated system, wherein the further robotic arm carries a navigation camera or other navigation modality, including possibly an in-body endoscopic camera. The movement of the further robotic arms is also controlled by the central control unit, thus providing robotic control of navigation/imaging, and placing all robotic arms, surgical and navigation, in the same coordinate system that is being controlled by the central control unit.

While the description and claims herein generally refer to the patient as “lying” on a surgical bed or table, the term “lying” is meant to embrace any and all positions that a patient might assume while undergoing a robotic surgical procedure, including lying prone on the bed or table, lying on a side on the bed or table, lying on a back on the bed or table, sitting on the table, bed or table, and the like.

The integrated multi-arm systems of the disclosed technology are useful for most robotic surgeries including but not limited to open surgeries, endoscopic and other minimally invasive surgery approaches. Any surgical application that will benefit from multiple robotic arms this, spinal surgeries, abdominal surgeries, gynecological surgeries, urological surgeries, and the like. In many or most cases, surgeries using the integrated surgical platforms of the disclosed technology will provide superior reachability and maneuverability by operating from a single, common robot coordinate system. The integrated surgical platforms disclosed herein can also be combined with more conventional navigation capabilities, e.g., using cameras mounted on a navigation arm and/or the deployment of endoscopic cameras. Thus, the integrated surgical platforms of the disclosed technology are useful for performing a full range of bone, joint, soft tissue and other surgeries.

### INCORPORATION BY REFERENCE

### Listing of Background Art

Commonly assigned US2023/0380916 has been described above and is incorporated by reference in its entirety. Multi-arm robotic systems positioned under a surgical table have been disclosed, for example in US2018/0193101 to Hashimoto. Bed-mounted multi-arm robotic surgical systems are described in US2010/0286712 to Won. WO2020/079596 to Zehavi discloses a non-mobile robotic surgery system incorporating multiple arms for imaging and optional tool deployment. However, the robotic arms are floor mounted, large and would thus suffer from inferior accuracy, be significantly disruptive of surgeon workflow, and will bear high costs.

The full disclosures of PCT Application PCT/______, (WSGR Docket No. 67551-711.602, Mathys Reference: P77437WO) entitled “SINGLE ORIGIN MARKER ASSEMBLIES AND METHODS FOR THEIR USE,” and PCT Application PCT/EP2024/052338, (WSGR Docket No. 67551-712.602, Mathys Reference: P77439WO) entitled “METHODS AND SYSTEMS FOR TRACKING MULTIPLE OPTICAL MARKERS IN A ROBOTIC SURGICAL PROCEDURE,” both of which are filed on the same day as the present application for the same applicant, are incorporated herein by reference in their entirety.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosed technology are set forth with particularity in the appended claims. A better understanding of the features and advantages of the disclosed technology will be obtained by reference to the following detailed description that sets forth illustrative embodiments, in which the principles of the invention are utilized, and the accompanying drawings of which:

FIGS. 1A and 1B are top views of a modular surgical robotic system with two robotic surgical carts prior to joining (FIG. 1A) and after they have been joined (FIG. 1B) into an integrated surgical robotic system, according to some embodiments of the disclosed technology.

FIG. 2 is perspective view of the integrated surgical robotic system of FIG. 1B in place beneath and across the width of a surgical bed with a draped patient, in accordance with some embodiments.

FIGS. 3A and 3B are top and side views of the integrated surgical robotic system of FIG. 1B in place beneath and across the width of a surgical bed with a draped patient, in accordance with some embodiments.

FIG. 4 is a top views of the integrated surgical robotic system of FIG. 1B being deployed in a spinal surgical procedure, in accordance with some embodiments.

FIG. 5 is a top views of the integrated surgical robotic system similar to that in FIG. 1B being deployed in a laparoscopic surgical procedure where the two carts are spaced further apart in a longitudinal direction, in accordance with some embodiments.

FIGS. 6A to 6C are schematic illustrations of different cart deployments, in accordance with some embodiments.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

With reference now to the figures and several embodiments, the following detailed description is provided.

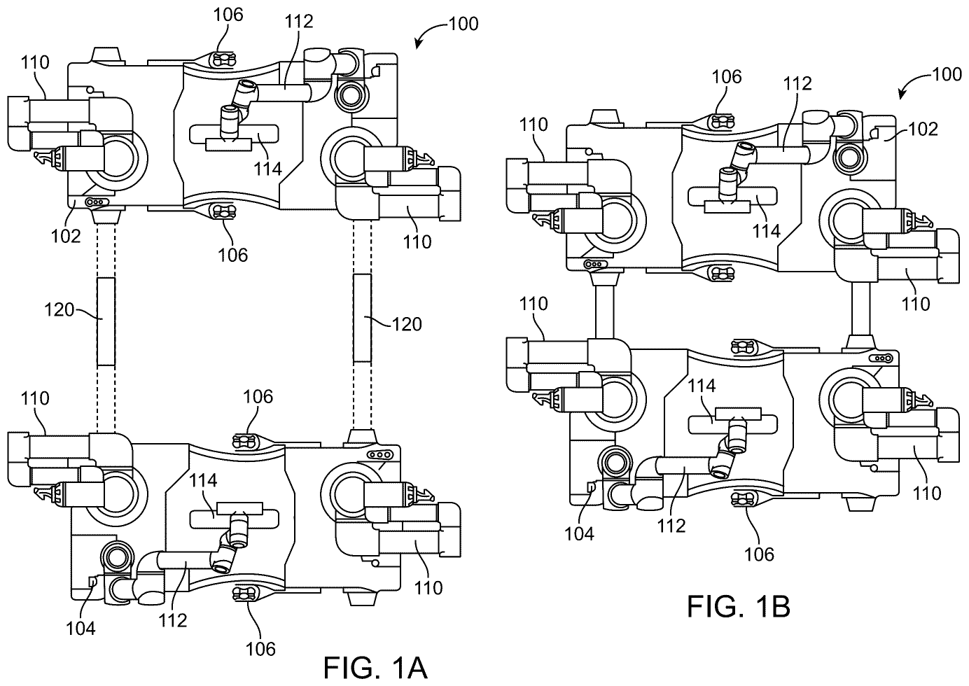

In some embodiments, as seen in FIGS. 1A and 1B, a surgical robotic system 100 according to the disclosed technology comprises first and second mobile surgical robotic carts 102 and 104. Each surgical robotic cart may be configured to be moved within a surgical operating room, typically having wheels, castors, rollers, or the like, that allow the robotic carts to be moved and positioned by operating room personnel. Wheels 106 are shown.

Each surgical robotic cart 102 and 104 may also have at least one and usually two or more “working” robotic arms 110 configured to hold and manipulate surgical tools (not shown). Some or all of the surgical robotic carts may typically also have a “surveillance” or “navigation” arm 112 which may hold a camera 114 or other sensor for scanning the surgical space during a robotic procedure, Additionally, some or all of the surgical robotic carts may have a controller carried internally and not visible in FIGS. 1A and 1B. Further, some or all of the surgical robotic carts may include a display and/or user interface allowing the surgeon to control aspects of the surgery while located at the surgical table. In some embodiments, the controller and/or display and interface can be located remotely from the carts. As described this far, the mobile carts 102 and 104 can be constructed similarly to those described in parent U.S. patent application Ser. No. 18/271,595, the full disclosure of which is incorporated herein by reference.

The first and second mobile surgical robotic carts 102 and 104 may differ from hose previously described in that they are configured to be separably joined by connectors 120 in a side-by-side manner, as shown in FIG. 1B. The connectors 120 may provide both a rigid mechanical link between the two carts 102 and 104 as well as provided, electrical connections, usually both low power connection for control and digital communication as well as high power for operating the robotic mechanical components. Data and control connectivity can be provided wirelessly, e.g., Bluetooth, WiFi, radio, or the like, and high power could be provided separately to each cart (they could be separately plugged in). The connection may be rigid so that, after being joined, the separate mobile carts 102 and 104 are held in a fixed positional relationship to provide an integrated surgical robotic platform having a single, common integrated surgical robotic platform, as described elsewhere herein in more detail.

The controller typically may comprise a computer, microprocessor, or the like, suitable for controlling and coordinating the movement of all robotic arms 110 and 112. Control of the robotic arms 112 and 114 may comprise, consist essentially of, or consist of, kinematic control where the controller tracks movement of at least some of the robotic arms bases primarily or solely on kinematically tracking movement of the arm, usually without optical tracking.

The controller may control the movement of the robotic arms within the common robotic surgical coordinate system of the integrated surgical robotic platform and may be thus able to deploy two or more surgical arms on either side of an operating table, as well as at least one navigation arm on one side of an operating table. Both cameras 114 can be used in many instances. The working surgical arms 110 can perform a wide range of surgical tasks because they have optimal accuracy and reachability, and the two navigation arms 112 may be able to deploy to optimal positions to assist in visualizing the surgical field from appropriate angles and elevations.

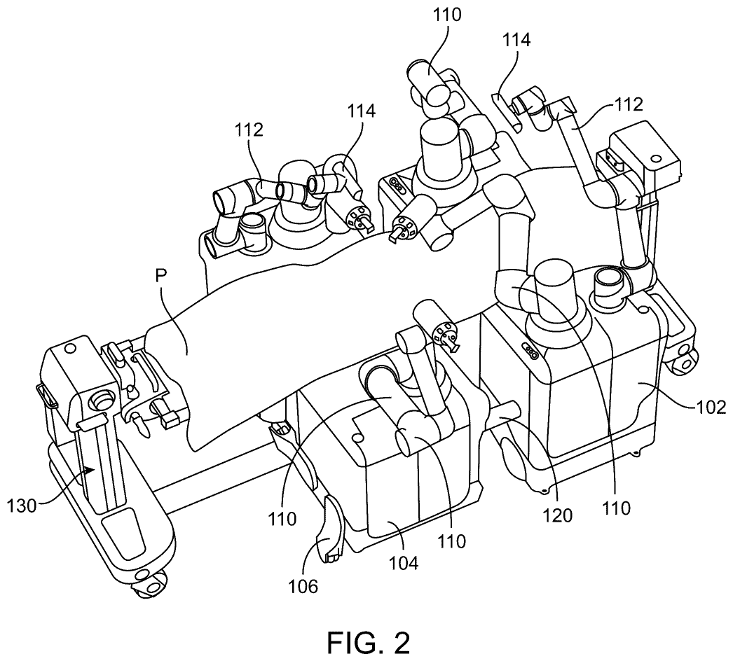

Referring now to FIG. 2, the surgical robotic system 100 of FIGS. 1A and 1B may be deployed under a surgical bed 130 by rolling the individual mobile surgical robotic carts 102 and 104 over the floor of the surgical suite so that they are located with one end on either side of the surgical bed. The individual surgical robotic carts 102 and 104 may be configured so that they may be advanced underneath the surgical bed and over any supporting structure which may be found under the bed. After the individual surgical robotic carts 102 and 104 are properly located, they can be joined by connecting the connectors 110 as shown in FIG. 1B. In some embodiments, however, the carts 102 and 104 can be joined prior to being positioned underneath the surgical bed 130 which may be generally more cumbersome.

As further shown in FIG. 2, the surgical robotic arms 110 and 112 may have bases that are spaced relatively far apart as compared to conventional multi-arm systems. For example, the surgical robotic arms 110 and 112 may be spaced apart by up to 80 cm and in some cases even up to 1 meter. By spacing the base of each robotic arm apart by a large distance, the arms may have increased or optimal reachability and maneuverability. Additionally, with the multiple surgical arms 110 and 112 being deployed on opposite sides of a surgical table, they each can be relatively small (as compared to conventional single-arm systems) and are more stable and thus more accurate.

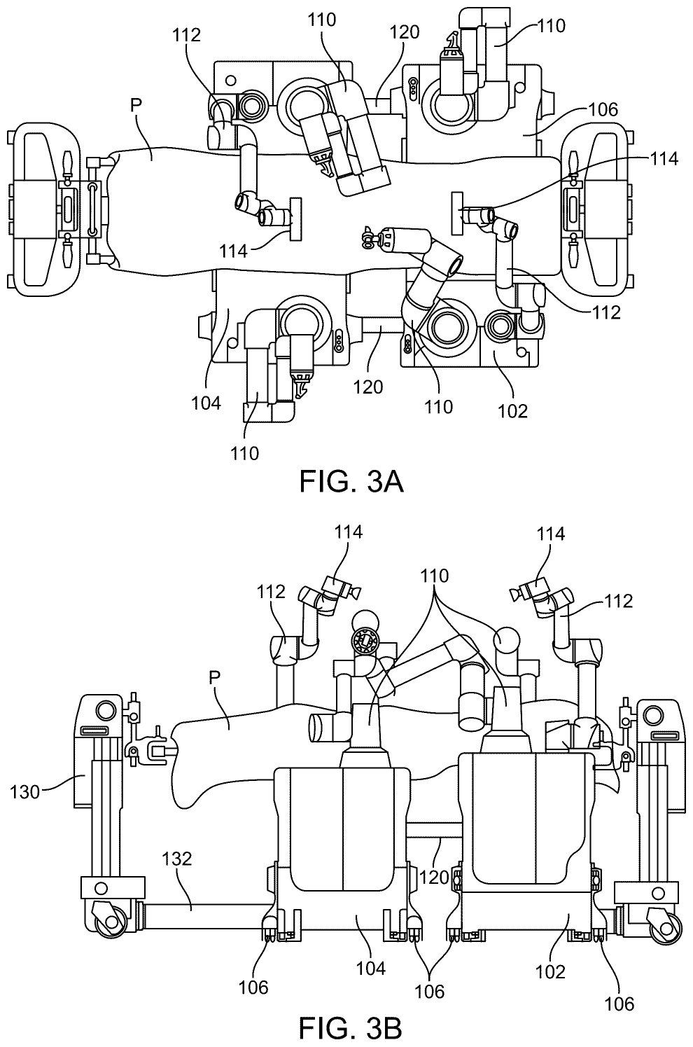

FIGS. 3A and 3B are top and side views, respectively, of the surgical robotic system 100 described previously with respect to FIGS. 1A, 1B, and 2. These views show a draped patient P on the bed undergoing A surgical procedure. FIG. 3B also shows how the carts may be located over his supporting beam 132 of the surgical bed.

One of skill in the art will easily understand that the constituent two or more multi-arm mobile surgical robotic carts of the present integrated system may be joined together by connection elements housing mechanical and electrical connections before the integrated unit is deployed under a surgical table. Further, the two or more constituent carts can be deployed under a surgical table and then joined together once in place. In either scenario, once joined, the constituent carts form an integrated multi-arm mobile surgical robotic system with a single coordinate system, wherein a central control unit can control and coordinate the movement of all of the robotic arms.

The embodiments of the disclosed technology may provide integrated multi-arm surgical robotic systems that are capable of being deployed and used in multiple different surgical applications. Solely by way of example, the disclosed technology may be used in general or soft-tissue surgery, whether open surgery or minimally invasive surgery. In some embodiments, one or more of the navigation arms of an integrated system according to an embodiment of the disclosed technology may hold an endoscopy camera suitable for insertion into a trocar or other access port as part of a minimally invasive procedure, while the surgical arms may hold and deploy suitable instruments for minimally invasive surgery. In this context, the disclosed system may provide added benefits over conventional multi-arm systems used for general, soft-tissue or minimally invasive surgery. Namely, the multiple arms of the disclosed system may be spaced far apart and, thus, have superior kinematics for the reasons already discussed. Also, the multiple arms can be deployed from various angles and can approach the surgical field from different and advantageous directions and trajectories, all while operating from a common coordinate system under the control and coordination of a central control unit that provides robotic control of surgical arms and robotic navigation (as compared to the teleoperated arrangements of most convention multi-arm systems). Thus, the disclosed system may provide for superior reachability, maneuverability, accuracy and kinematics as compared to known systems such as da Vinci.

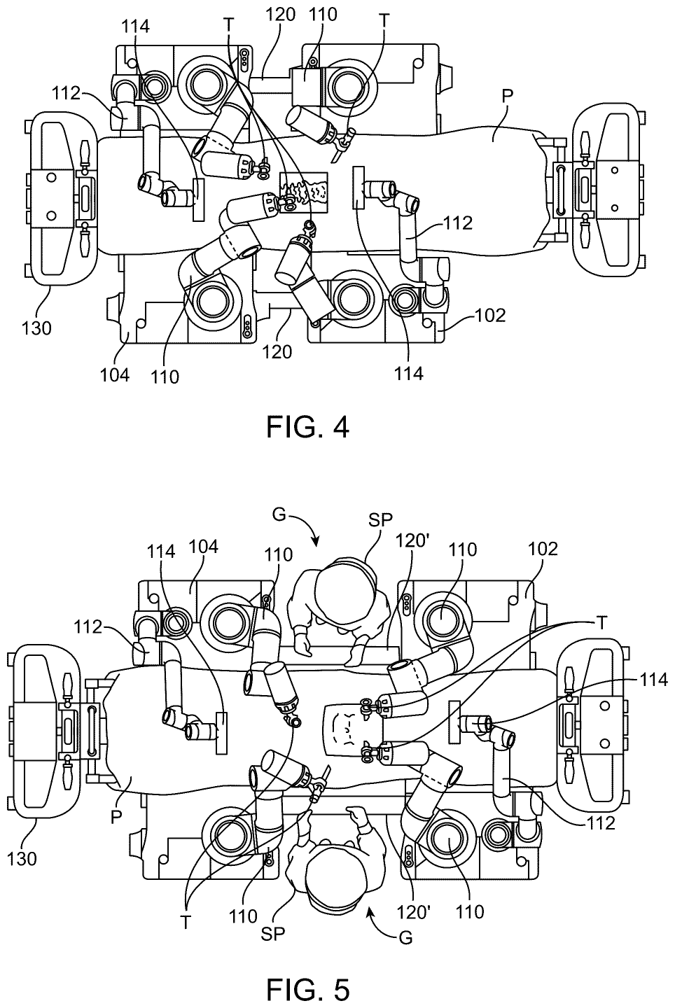

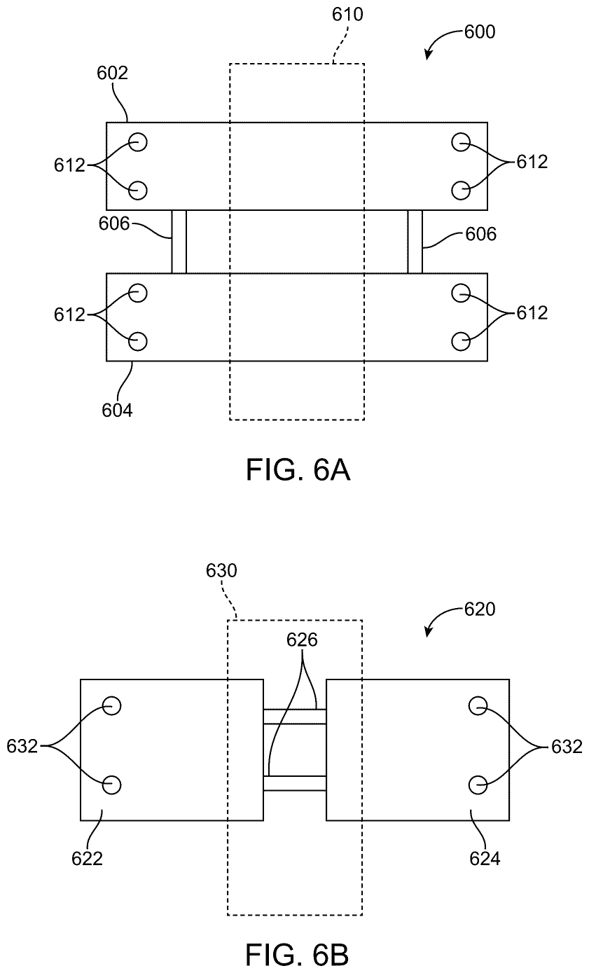

FIG. 4 shows the surgical robotic system 100 deployed for use in a spinal surgical procedure, in accordance with some embodiments. The mobile surgical robotic carts 102 and 104 may be separably connected and positioned beneath the patient bed as previously described. Relatively short connectors 120 may be used so that the carts 102 and 104 can be co-located toward and end of the bed. In some embodiments, in surgeries, the connectors may vary in length to achieve are spacings and robotic arm arrangements, as shown in FIG. 5 discussed below.

The working surgical arms 110 may be controlled by the controller in the common robotic surgical coordinate system and hold trocars T for providing access for tools to perform spinal surgery. For example, the trocars may be used to provide suitable access to portions of the spinal anatomy 614, such as disc spaces. In some embodiments, the other surgical arms could hold surgical tools that can be advanced through the trocars into the surgical field or could hold trocars to provide for other tool or camera access.

As further shown in FIG. 4, each of the navigation arms 112 may hold a navigation camera 114. The navigation arms 112 may be controlled by the controller in the common robotic surgical coordinate system in the same manner as the working robotic arms 110. The skilled artisan will understand that any conventional navigation camera is suitable for use in these embodiments or in any of the embodiments of the disclosed technology. In some embodiments, the navigation arms are under central robotic control since they are based in the integrated robotic platform as the surgical working arms 110. The navigation cameras 114 held by the navigation arms 112 may provide for any required view and tracking of the surgical field and patient anatomy using known and suitable markers and known techniques.

FIG. 5 shows two surgical personnel SP performing a minimally invasive abdominal surgery procedure on a patient P using the surgical robotic system 100, in accordance with some embodiments. A primary difference in the set-up of FIG. 5 compared to that of FIG. 4 is that the mobile surgical carts 102 and 104 are space sufficiently far apart in a longitudinal direction that a gap B allows the surgical personnel to stand closer to the patient P between the portions of the mobile surgical carts that extend outwardly from beneath the table 130. This can be achieved, for example by using longer connectors 120′ as illustrated. Trocars T are shown at various degrees of deployment into the abdomen of the patient P.

The ability to space the mobile surgical arts 102 and 104 at a greater distance while maintaining all surgical arms 110 and 112 in a common robotic surgical coordinate system is an advantage that allows optimal spacing between the robotic arms as described previously. Different spacing between the mobile carts 102 and 104 may be chosen to accommodate user preference or, in some cases, for purposes of operator access during particular surgical procedures.

The skilled artisan will appreciate that any suitable minimally invasive surgical tool can be inserted into the abdominal surgical field through any of the trocars that are deployed into the patient's abdomen. It will also be easily understood that, while conventional navigation cameras 114 are shown in FIG. 5 and held at a conventional distance, the navigation arms 112 can also hold endoscopic cameras (not shown) instead of the navigation cameras, and that the endoscopic cameras could be inserted into the abdominal surgical field through the trocars that have been deployed into the patient's abdomen. Embodiments are also possible where different numbers of surgical and navigation arms are deployed from each mobile cart to facilitate different surgical approaches. Solely by way of example, but not by way of limitation, some minimally invasive surgical procedures could benefit from conventional navigation cameras maintaining a view of, for example, surface markers, along with endoscopic cameras maintaining an advantageous view of a patient's internal anatomy. Thus, an embodiment of the disclosed technology could easily be envisioned wherein each constituent mobile cart comprises two surgical arms and two navigation/imaging arms. In some embodiments, the two surgical arms might hold trocars with surgical tools inserted into a patient's abdomen. One of the navigation arms might hold an additional trocar with an endoscopic camera inserted into the abdominal surgical field and the other navigation arm might hold a conventional navigation camera.

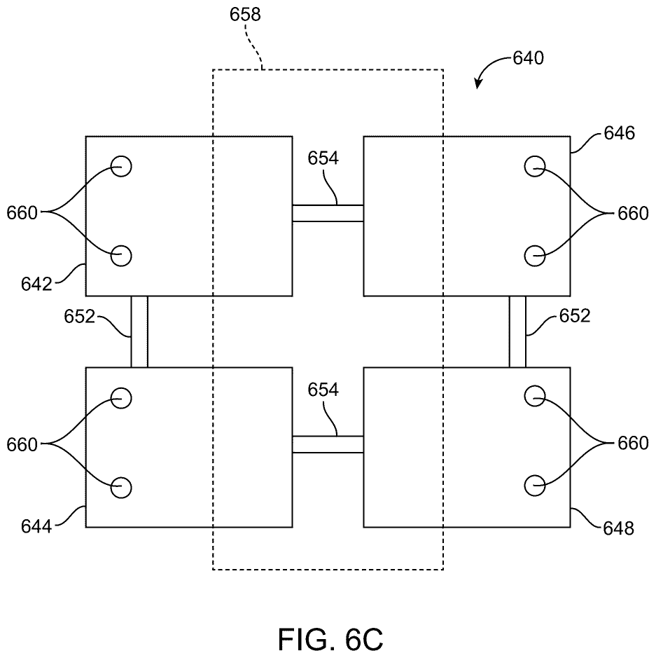

Referring to FIGS. 6A to 6C, the individual mobile carts of the surgical robotic system of the disclosed technology can be separably joined in a variety of different patterns and configurations, in accordance with some embodiments. As shown in FIG. 6A, a surgical robotic system 600 may comprise a first mobile surgical robotic cart 602 and a second mobile surgical robotic cart 604. The carts 602 and 604 may be elongated, each having a length greater than a width, where the length is sufficient to extend across and beyond each side of a surgical bed 610 and the width is a fraction of the length of the surgical bed, allowing two or more carts to be positioned side-by-side beneath the surgical bed. The surgical robotic carts 602 and 604 may be positioned with their lengths extending transversely across and beneath the width of the surgical bed 610 and may be attached side-by-side using connectors 606. In this way, a plurality of surgical robots 612 can be positioned on both sides of the surgical bed 610 and the spacing of the robots can be changed as needed by controlling the distance between the adjacent robotic carts 602 and 604, e.g., by using connectors having different lengths. The arrangement shown in FIG. 6A may be achieved by the surgical robotic system 100 previously described. An advantage of this arrangement is that the axial distance between each pair of surgical robots 612 can be adjusted by choosing connectors 606 having different lengths. Thus, the arrangement of surgical robots can be tailored to any particular surgical procedure that is being performed.

Referring to FIG. 6B, surgical robotic system 620 comprises a first mobile surgical robotic cart 622 and a second mobile surgical robotic cart 624. In contrast to the surgical robotic system 600 described above, the mobile carts 622 and 624 of surgical robotic systems 620 may be configured to extend only partially beneath a surgical bed 630 where they are separably joined by connectors 626. While prior art systems are known to place independent mobile surgical carts on either side of a patient bed, those carts typically operate independently and have separate robotic surgical coordinate systems. By providing connectors 626 which hold the surgical robotic carts 622 and 624 in a fixed positional relationship, the surgical robotic system 620 allows all surgical robotic arms 632 to operate in a common, single robotic surgical coordinate system, providing precise and accurate kinematic control of the robotic arms using a single, common controller (not shown). An advantage of this arrangement is that the lateral distance between each pair of surgical robots 632 can be adjusted by choosing connectors 626 having different lengths. Thus, the arrangement of surgical robots can be tailored to any particular surgical procedure that is being performed.

Referring now to FIG. 6C, a surgical robotic system 640 comprises a first mobile surgical robotic cart 642, a second mobile surgical robotic cart 644, a third mobile surgical robotic cart 646, and a fourth mobile surgical robotic cart 648. The carts 642-648 may be similar to carts 622 and 624 in surgical robotic system 620 in that they are configured to extend only partially beneath a surgical bed 658. They differ from the previously described carts, however, in that they are configured to join adjacent carts in both axial and lateral directions. As illustrated, axial connector 652 can be used to rigidly attach axially adjacent carts while lateral connectors 654 can be used to join laterally adjacent carts. The resulting integrated robotic platform can include four or more individual mobile carts which can be separately joined into a two-dimensional array of carts where both the axial and lateral distances between each cart can be changed by selecting the length of either of both the axial and lateral connectors 652 and 654. This is advantageous in that the distance between surgical robots 660 carried by each of the carts can be adjusted in both the axial and lateral dimensions, allowing great flexibility in arranging robot positions for performing a particular robotic surgical procedure.

### Glossary

Certain terms and phrases as used in the specification and claims herein are defined as follows:

The term chassis refers to a frame or structure which provides the base or platform for a surgical robotic arm. The chassis will usually carry all motors, electrical systems, pneumatic system, and other components needed to move the robotic arms and drive tools carried by the arms, if necessary.

The term connector refers to a structure that rigidly couples adjacent robotic chassis to form an integrated robotic platform.

The term fixed and phrase fixed positional relationship mean that chassis or other system components do not move relative to each other although they may move or be moved in tandem.

The phrase integrated robotic platform means the assembly of two, three, four, or more surgical robotic chassis in a manner that allows all robotic arms carried by the chassis to be moved in a common robotic surgical coordinate system.

The term rigid means that a structure, such as the chassis, will retain its shape in ordinary use. While all structures are flexible to some minimum extent, the rigid chassis and other structures herein will resist deformation so that they will provide a stable platform for the robotic arms.

The phrase rigidly couple means that independent chassis and other structures can be coupled so that when joined, they provide a common rigid base that provides a stable platform for all arms on all joined chassis. In particular, the arms on rigidly coupled chassis and carts may be moved within a single, common robotic surgical coordinate system.

The phrase robotic surgical coordinate system means the coordinate system used to control movement of the surgical robotic arms relative to the chassis which carries the arms, usually being a Cartesian coordinate system or a polar coordinate system.

The phrase common robotic surgical coordinate system means the coordinates system used to move all robotic arms in an integrated robotic platform, typically using a single controller.

The phrase separably joined means that separate chassis or other independent system components may be joined together in a fixed positional relationship and thereafter detached from each other so that they once again are separate and usually in the same condition that they were in before being joined. Such joining usually includes both mechanically and electrically linking the chassis in a manner that allows repeated connection and disconnection so that the chassis can be connected after they are positioned at a patient bedside prior to a procedure and discontinued and repositioned or removed after completion of the procedure.

### NUMBERING IN DRAWINGS

100 surgical robotic system
102 first mobile surgical robotic cart
104 second mobile surgical robotic cart
106 wheels
110 working robotic arms
112 surveillance/navigation robotics arms
114 cameras
120 connectors
130 surgical bed
600 surgical robotic system
602 first mobile surgical robotic cart
604 second mobile surgical robotic cart
606 connectors
610 surgical bed
612 surgical robots
620 surgical robotic system
622 first mobile surgical robotic cart
624 second mobile surgical robotic cart
626 connectors
630 surgical bed
632 surgical robots
640 surgical robotic system
642 first mobile surgical robotic cart
644 second mobile surgical robotic cart
646 third mobile surgical robotic cart
648 fourth mobile surgical robotic cart
652 axial connectors
654 lateral connectors
658 surgical bed
660 surgical robots

One of skill in the art will realize that the embodiments described herein are representative in nature. Variations on the disclosed embodiments are possible while staying within the bounds of the disclosed technology. Solely by way of example, the skilled artisan will understand that constituent mobile carts of the current integrated system could provide three or more surgical arms, in addition to one or more navigation arms, while still staying within the spirit of the disclosed technology. One of skill in the art will also understand that an embodiment of an integrated multi-arm mobile surgical robotic system according to the disclosed technology could incorporate three or more constituent mobile robotic carts joined together by similar connection elements as those shown here. To form an integrated system, the embodiments all share a common coordinate system and a central control unit that controls and coordinates the movements of the provided surgical and navigation arms. While some embodiments of the disclosed technology have been shown and described herein, it will be obvious to those skilled in the art that such embodiments are provided by way of example only. Numerous variations, changes, and substitutions will now occur to those skilled in the art without departing from the scope of the disclosed technology. It should be understood that various alternatives to the embodiments of the invention described herein may be employed in practicing the invention. It is intended that the following claims define the scope of the disclosed technology and that methods and structures within the scope of these claims and their equivalents be covered thereby.

What is claimed is:

1. A modular surgical robotic system for performing robotic surgery on a patient lying on a surgical bed, the modular surgical robotic system comprising:
a first robotic chassis and a second robotic chassis, wherein the first and second robotic chassis are configured to be separably joined to form an integrated robotic platform having a common robotic coordinate system relative to the surgical bed;
one or more robotic arms disposed on each of the first robotic and second robotic chassis; and
a controller configured to robotically coordinate the movement of each of the robotic arms in the common coordinate system.

2. The modular surgical robotic system of claim 1, wherein the first and second robotic chassis are configured to be positioned on opposite sides of the surgical bed and to be separably joined beneath and across a width of the surgical bed. to form the integrated robotic platform

3. The modular surgical robotic system of claim 2, further comprising a third robotic chassis and a fourth robotic chassis configured to be positioned on opposite sides of the surgical bed and to be separably joined beneath and across a width of the surgical bed, the third and/or fourth robotic chassis being further configured to be separably joined to the first and/or the second robotic chassis to form an integrated platform having a common robotic coordinate system for all four robotic chassis.

4. The modular surgical robotic system of claim 1, wherein the first and second robotic chassis are each configured to be positioned beneath and across a width of the surgical bed and to be separably joined in a longitudinal direction.

5. The modular surgical robotic system of claim 1, wherein each of the robotic chassis is configured to be moved and repositioned relative to the surgical bed.

6. The modular surgical robotic system of claim 2, wherein the first and second robotic chassis each comprise rollers that allow the first and second chassis to be moved over a floor.

7. The modular surgical robotic system of claim 1, further comprising connectors configured to separably join adjacent pairs of robotic chassis in a fixed relationship.

8. The modular surgical robotic system of claim 7, wherein the connectors comprise electrical conductors for power and/or data transmission between the adjacent pairs of robotic chassis.

9. The modular surgical robotic system of claim 1, wherein the robotic arms each have a point of origin and wherein the points of origin of at least two of the robotic arms are located at least 80 cm apart from each other, usually at least one meter apart from each other.

10. The modular surgical robotic system of claim 1, wherein the first and second robotic chassis are configured to be separably joined before deployment under a surgical table.

11. The modular surgical robotic system of claim 1, wherein the first and second robotic chassis are configured to be separably joined after deployment under a surgical table.

12. The modular surgical robotic system of claim 1, wherein the one or more robotic arms comprise both surgical arms configured to hold surgical tools and surveillance and/or navigation arms configured to hold sensors.

13. A method for arranging and controlling a surgical robotic system to perform robotic surgery on a patient lying on a surgical bed having two sides in a surgery room, the method comprising:
separably joining a first robotic chassis and a second robotic chassis to form an integrated robotic platform having a common robotic coordinate system, wherein each of the robotic chassis carries one or more robotic arms;
positioning the integrated surgical platform beneath and across a width of the surgical bed to locate the one or more robotic arms on both sides of the surgical bed; and
controlling movement of the one or more robotic arms in the common robotic coordinate system using a common controller.

14. The method of claim 13, wherein the first robotic chassis and the second robotic chassis are separably joined prior to positioning the integrated surgical platform beneath and across a width of the surgical bed.

15. The method of claim 14, further comprising separably joining and positioning third and fourth robotic chassis to the first and/or the second robotic chassis beneath and on opposite sides of the surgical bed to be part of the integrated platform having a common robotic coordinate system.

16. The method of claim 13, wherein the first and second robotic chassis are each positioned beneath and across a width of the surgical bed and separably joined in a longitudinal direction.

17. The method of claim 13, wherein the first robotic and second robotic chassis are separably joined after positioning the integrated surgical platform beneath and across a width of the surgical bed.

18. The method of claim 13, wherein the first robotic and second robotic chassis are separably joined before positioning the integrated surgical platform beneath and across a width of the surgical bed.

19. The method of claim 13, wherein positioning comprises moving the robotic chassis over a floor surface adjacent to the surgical bed.

20. The method of claim 13, wherein the robotic chassis comprise rollers and moving comprises manually pushing the chassis over the floor.
